(19) United States
(12) Patent Application Publication
(10) Pub. No.: US 2016/0135745 A1
(43) Pub. Date: May 19, 2016

(54) MONITORING GLOVE AND SYSTEM

(71) Applicant: Winbond Electronics Corp., Taichung City (TW)

(72) Inventors: Chia-Chi CHANG, Taipei City (TW); Tzu-Chien HSIAO, Zhubei City (TW)

(21) Appl. No.: 14/547,648

(22) Filed: Nov. 19, 2014

(51) Int. Cl. A61B 5/00; A61B 7/04; A61B 5/1455; A61B 5/0205; A61B 5/0408

(52) U.S. Cl. CPC A61B 5/6806; A61B 5/02055; A61B 5/04085; A61B 5/14552; A61B 5/742; A61B 5/0002; A61B 7/04; A61B 5/024

(57) ABSTRACT

A measuring glove including a first sensing unit and a second sensing unit is provided. The first sensing unit measures a first physiological signal. The second sensing unit measures a second physiological signal. When the first sensing unit operates, the second sensing unit stops operating. When the second sensing unit operates, the first sensing unit stops operating.

### BACKGROUND OF THE INVENTION

1. Field of the Invention

The invention relates to a monitoring glove, and more particularly to a monitoring glove monitoring physiological symptoms.

2. Description of the Related Art

Physiological monitors are common medical apparatuses to measure physiological parameters. Doctors and nurses diagnose and treat sick people according to measured physiological indexes. However, different physiological indexes (e.g. electrocardiogram (ECG), blood pressure, temperature and respiration) are captured by different physiological monitors. The complexity of measurement is increased.

### BRIEF SUMMARY OF THE INVENTION

In accordance with an embodiment, a measuring glove comprises a first sensing unit and a second sensing unit. The first sensing unit measures a first physiological signal. The second sensing unit measures a second physiological signal. When the first sensing unit operates, the second sensing unit stops operating. When the second sensing unit operates, the first sensing unit stops operating.

In accordance with another embodiment, a measuring system comprises a glove and an external device. The glove comprises a first sensing unit, a second sensing unit and a transmittal unit. The first sensing unit measures a first physiological signal. The second sensing unit measures a second physiological signal. When the first sensing unit operates, the second sensing unit stops operating. When the second sensing unit operates, the first sensing unit stops operating. The transmittal unit transmits the first and second physiological signals. The external device receives the first and second physiological signals.

A detailed description is given in the following embodiments with reference to the accompanying drawings.

### BRIEF DESCRIPTION OF THE DRAWINGS

The invention can be more fully understood by referring to the following detailed description and examples with references made to the accompanying drawings, wherein:

FIG. 1 is is a schematic diagram of an exemplary embodiment of a measuring system, in accordance with some embodiments;

FIGS. 2A˜2F are position schematic diagrams of exemplary embodiments of sensing units, in accordance with some embodiments.

### DETAILED DESCRIPTION OF THE INVENTION

The making and using of the embodiments of the disclosure are discussed in detail below. It should be appreciated, however, that the embodiments provide many applicable inventive concepts that can be embodied in a wide variety of specific contexts. The specific embodiments discussed are merely illustrative, and do not limit the scope of the disclosure.

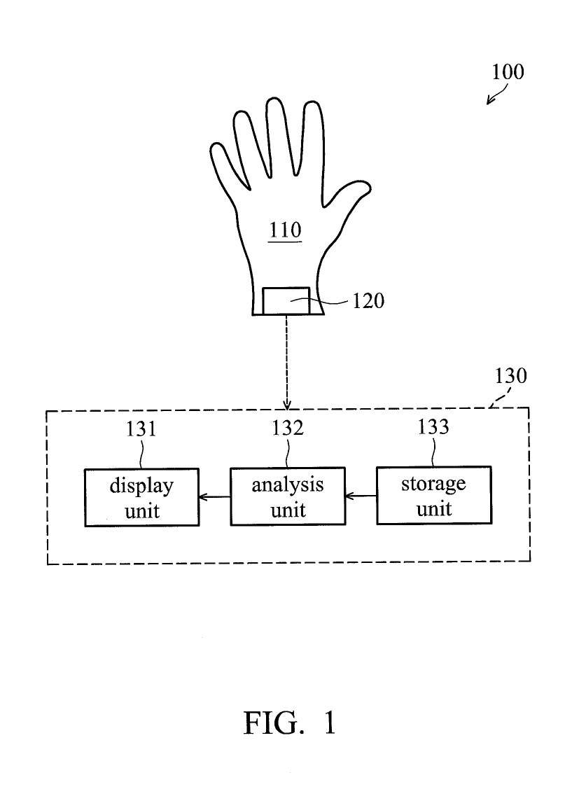

FIG. 1 is a schematic diagram of an exemplary embodiment of a measuring system, in accordance with some embodiments. The measuring system 100 comprises a measuring glove 110, a transmittal unit 120 and an external device 130. In this embodiment, the measuring glove 110 comprises at least two sensing units to measure at least two physiological signal. The invention does not limit the types of physiological signals. In one embodiment, the physiological signals measured by the measuring glove 110 comprise at least two of a heart rate signal, an ECG signal, a SpO₂ signal, a temperature signal and a pulse signal. The SpO₂ refers to the percentage of oxygen in a pilot's blood stream.

In this embodiment, the sensing units do not operate simultaneously. Assuming that the measuring glove 110 comprises a first sensing unit and a second sensing unit, while the first sensing unit operates, the second sensing unit does not operate. Similarly, during the operation of the second sensing unit, the first sensing unit does not operate. The invention does not limit the kind of first and second sensing units. In one embodiment, the first and second sensing units are the same. For example, each of the first and second sensing units is a photo sensor, a voltage sensor, a vibration sensor, a pressure sensor, or an audio sensor.

In other embodiments, the first sensing unit is different from the second sensing unit. For example, the first sensing unit is a photo sensor, a voltage sensor, a vibration sensor, a pressure sensor, or an audio sensor, and the second sensing unit is another from the above list.

In this embodiment, the physiological signal generated from the first sensing unit is different from the physiological signal generated from the second sensing unit. For example, the physiological signal generated from the first sensing unit is one of a heart rate signal, a ECG signal, a SpO₂ signal, a temperature signal and a pulse signal, and the physiological signal generated from the second sensing unit is another of the heart rate signal, the ECG signal, the SpO₂ signal, the temperature signal and the pulse signal.

In one embodiment, a medical technician wears the measuring glove 110 and contacts with a patient to measure physiological condition. The measuring glove 110 is covered with a specific consumable film for hygiene. After measuring, medical technician may replace the film on the surface of the measuring glove 110.

After obtaining physiological signals, the transmittal unit 120 transmits the physiological signals to the external device 130. The invention does not limit how the transmittal unit 120 transmits the physiological signals. The transmittal unit 120 may be utilize a wired transmission technology or a wireless transmission technology to transmit the physiological signals. Assume that the transmittal unit 120 transmits the physiological signals to various external devices. In some embodiments, the transmittal unit 120 transmits a portion of the physiological signals to an external device according to a wireless transmission technology and transmits another portion of the physiological signals to another external device according to a wired transmission technology. In other embodiment, the transmittal unit 120 transmits the physiological signals to remote devices via the Internet. Therefore, the convenience of the measuring system is increased.

The external device 130 receives and analyzes the physiological signals. The medical technician obtains the physiological condition of the patient according to the analysis result generated by the external device 130. In one embodiment, the external device 130 comprises a display unit 131, an analysis unit 132 and a storage unit 133, but the disclosure is not limited thereto. In this embodiment, the analysis unit 132 analyzes the physiological signals provided by the transmittal unit 120 to generate an analysis result. The display unit 131 displays text or image to present the analysis result. The storage unit 133 stores the analysis result.

In other embodiments, the external device 130 may only comprise a display unit 131 and an analysis unit 132. In another embodiment, the external device 130 only comprises an analysis unit 132 and a storage unit 133. The invention does not limit the kind of external device 130. In one embodiment, the external device 130 is a smartphone, a computer or a cloud server.

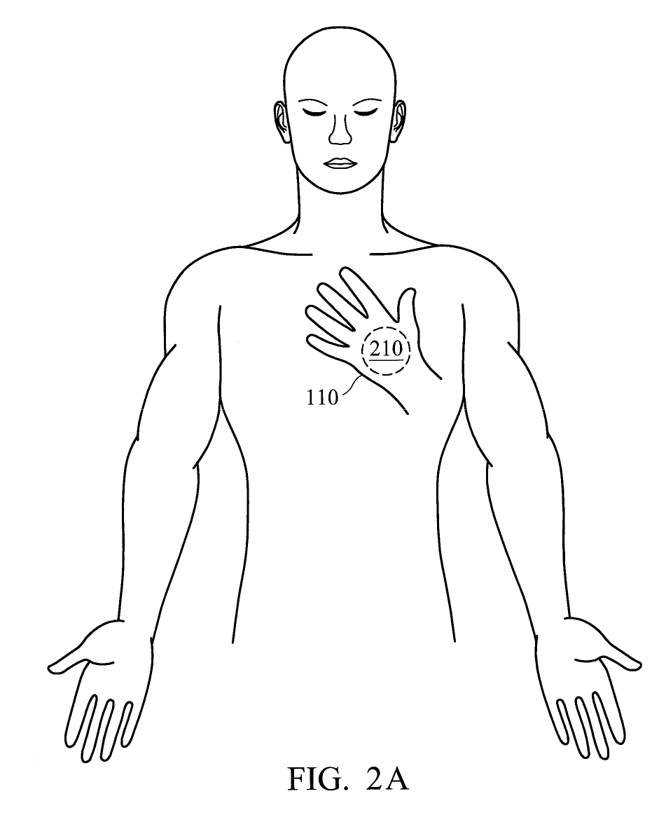

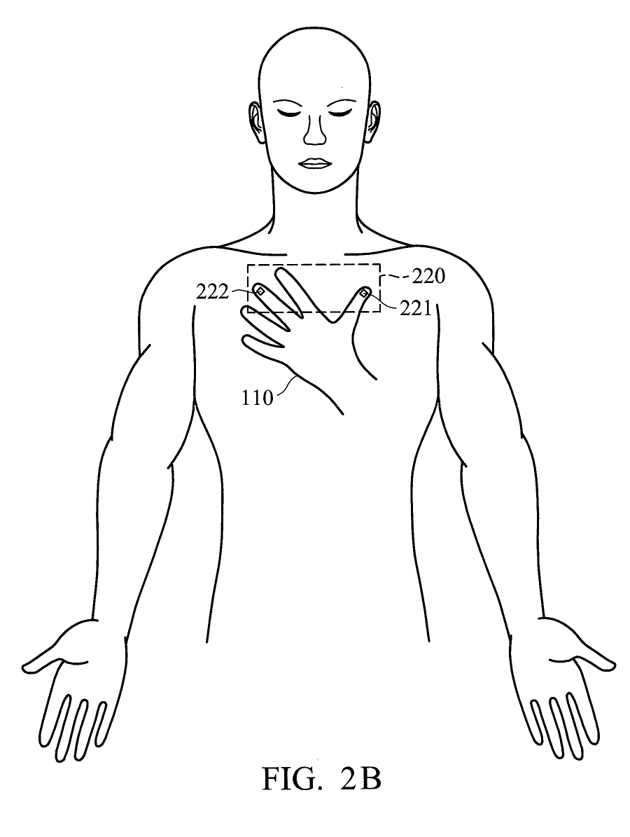

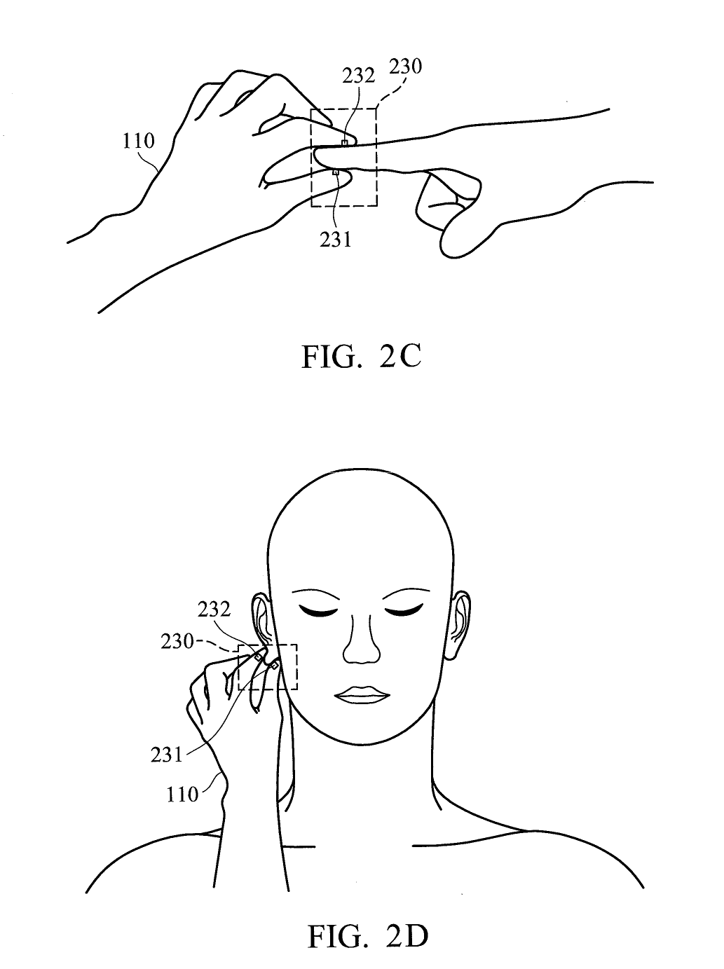

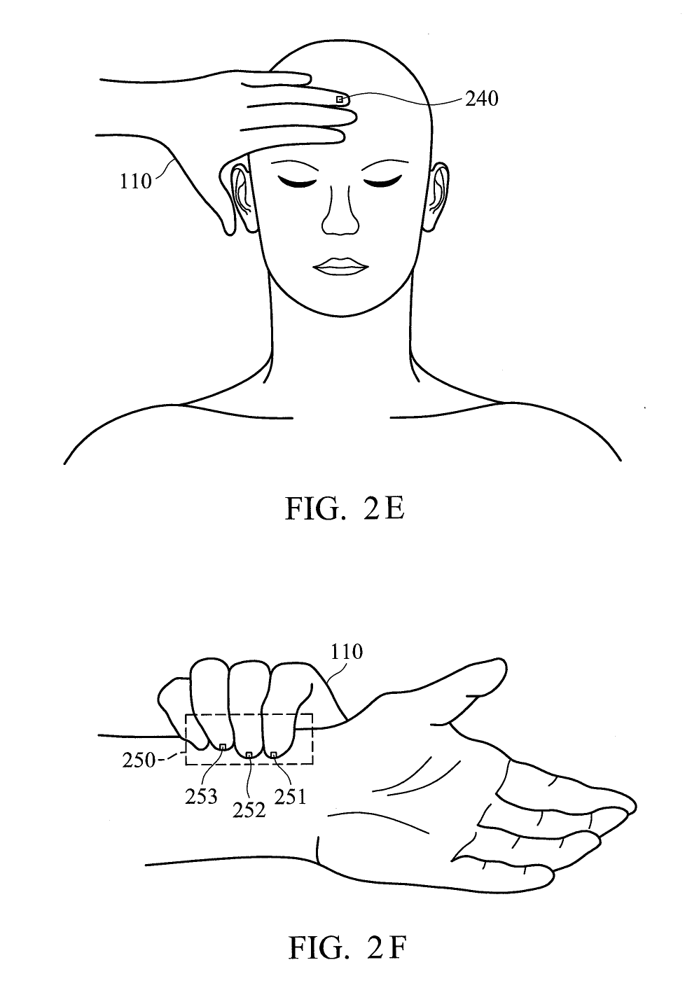

The invention does not limit the positions of the sensing units. The sensing unit can be disposed in any position of the glove. FIGS. 2A-2F are position schematic diagrams of exemplary embodiments of sensing units, in accordance with some embodiments. Refer to FIG. 2A, the measuring glove 100 comprises a sensing unit 21 in this embodiment 0. The sensing unit 210 is disposed in the palm portion of the measuring glove 100 to measure heart rate. In one embodiment, the medical technician wears the measuring glove 100 and contacts the anterior chest or the posterior chest of the patient by the palm portion of the measuring glove 100 to measure the heart sound of the patient. In one embodiment, the sensing unit 210 is a vibration sensor or an audio sensor, such as microphone.

Refer to FIG. 2B, in this embodiment, the measuring glove 100 comprises a sensing unit 220. The sensing unit 220 comprises electrodes 221 and 222 to measure the physiological condition of a patient. In this embodiment, the electrode 221 is disposed in the thumb portion of the measuring glove 100, and the electrode 222 is disposed in a middle finger portion of the measuring glove 100. The medical technician wears the measuring glove 100 and contacts left and right infraclavicular fossa of the patient by the thumb and middle finger portions of the measuring glove 100 to obtain a ECG signal. In one embodiment, the sensing unit 220 is a voltage sensor.

As shown in FIG. 2C, the measuring glove 100 comprises a sensing unit 230. The sensing unit 230 comprises a light receiver 231 and a light emitter 232. The light emitter 232 is disposed in the thumb portion of the measuring glove 100 to emit light. The light receiver 231 is disposed in the first finger portion of the measuring glove 100 to receive transmitted light.

The medical technician wears the measuring glove 100 and grips the ear or the finger of the patient by the first finger and the thumb portions of the measuring glove 100. When the light emitter 232 emits a light, the light receiver 231 generates a SpO₂ signal according to the light emitting through the ear or the finger. In one embodiment, the light is a red light or an infrared light.

Refer to FIG. 2C, the light emitter 232 is disposed in a first side of a finger of a patient, and the light receiver 231 is disposed in a second side of the finger of the patient. In this embodiment, the first side is opposite to the second side. In FIG. 2D, the light emitter 232 is disposed in one side of an ear of a patient and the light receiver 231 is disposed in another side of the ear of the patient.

Refer to FIG. 2E, in this embodiment, the measuring glove 100 comprises a sensing unit 240. The sensing unit 240 is a temperature sensor and disposed in a ring finger portion of the measuring glove 100. The medical technician wears the measuring glove 100 and contacts the forehead of a patient by the ring finger portion of the measuring glove 100 to obtain a temperature signal.

In FIG. 2F, the measuring glove 100 comprises a sensing unit 250. The sensing unit 250 comprises sensors 251-253. The sensor 251 is disposed in the first finger portion of the measuring glove 100. The sensor 252 is disposed in the middle finger portion of the measuring glove 100. The sensor 253 is disposed in the ring finger portion of the measuring glove 100. The medical technician wears the measuring glove 100 and contacts the wrist of a patient by the first, middle and ring finger portions to measure the pulse of the patient.

In one embodiment, the sensors 251-253 are pressure sensors. In other embodiments, the sensing unit 250 only comprises a pressure sensor disposed in the first, middle or ring finger portion of the measuring glove 100. In this case, the medical technician utilizes one finger portion of the measuring glove 100 to obtain the pulse of the patient.

In this embodiment, the measuring glove 100 comprises at least two of the sensing units 210, 220, 230, 240 and 250 to measure two physiological condition, such as temperature and pulse. In other embodiment, the measuring glove 100 comprises sensing units 210, 220, 230, 240 and 250. When one of the sensing units 210, 220, 230, 240 and 250 operates, other sensing units do not operate. In some embodiments, the measuring glove 100 measures SpO₂ of the patient according to light transmission technology.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A measuring glove, comprising:
a first sensing unit measuring a first physiological signal; and
a second sensing unit measuring a second physiological signal, wherein when the first sensing unit operates, the second sensing unit stops operating, and when the second sensing unit operates, the first sensing unit stops operating.

2. The measuring glove as claimed in claim 1, wherein the first sensing unit is one of a light sensor, a voltage sensor, a vibration sensor, a pressure sensor, and an audio sensor, and the second sensing unit is another of the light sensor, the voltage sensor, the vibration sensor, the pressure sensor, and the audio sensor.

3. The measuring glove as claimed in claim 1, wherein the first physiological signal is one of a heart sound signal, an ECG signal, a SpO₂ signal, a temperature signal and a pulse signal, and the second physiological signal is another of the heart sound signal, the ECG signal, the SpO₂ signal, the temperature signal and the pulse signal.

4. The measuring glove as claimed in claim 1, wherein the first sensing unit comprises a light emitter and a light receiver.

5. The measuring glove as claimed in claim 4, wherein the light emitter is disposed in a first side of a finger or a ear of a patient, the light receiver is disposed in a second side of the finger or the ear of the patient, and the first side is opposite to the second side.

6. The measuring glove as claimed in claim 5, wherein the light emitter emits light emitting through the finger or the ear, the light receiver generates the first physiological signal according to the light emitting through the finger or the ear.

7. The measuring glove as claimed in claim 4, further comprising:
a thumb portion configured to dispose the light receiver; and
a first finger portion configured to disposed the light emitter.

8. The measuring glove as claimed in claim 1, further comprising:
a palm portion configured to dispose the first sensing unit.

9. The measuring glove as claimed in claim 1, further comprising:
a thumb portion configured to dispose the first sensing unit; and
a middle finger portion, wherein the first sensing unit is an ECG sensor, and the ECG sensor comprises a first electrode disposed in the thumb portion and a second electrode disposed in the middle finger portion.

10. The measuring glove as claimed in claim 1, further comprising:
a ring finger portion configured to dispose the first sensing unit, wherein the first sensing unit is a temperature sensor.

11. The measuring glove as claimed in claim 1, further comprising:
a first finger portion;
a middle finger portion; and
a ring finger portion, wherein the first sensing unit is a pressure sensor disposed in the first finger portion, the middle finger portion and the ring finger portion.

12. The measuring glove as claimed in claim 1, further comprising:
a film covering the first sensing unit.

13. The measuring glove as claimed in claim 1, further comprising:
a third sensing unit measuring a third physiological signal;
a fourth sensing unit measuring a fourth physiological signal;
a fifth sensing unit measuring a fifth physiological signal, wherein when one of the first, second, third, fourth and fifth sensing units operates, another sensing units do not operate.

14. The measuring glove as claimed in claim 13, further comprising:
a palm portion configured to dispose the first sensing unit;
a thumb portion;
a first finger portion; a middle finger portion; and
a ring finger portion, wherein the second sensing unit is disposed in the thumb and middle finger portions, the third sensing unit is disposed in the thumb and the first finger portions, the fourth sensing unit is disposed in the ring finger portion, and the fifth sensing unit is disposed in the first, middle and ring finger portions.

15. The measuring glove as claimed in claim 11, further comprising:
a transmittal unit transmitting the first and second physiological signals to an external device.

16. A measuring system comprising:
a glove comprising:
a first sensing unit measuring a first physiological signal;
a second sensing unit measuring a second physiological signal, wherein when the first sensing unit operates, the second sensing unit stops operating, and when the second sensing unit operates, the first sensing unit stops operating; and
a transmittal unit transmitting the first and second physiological signals; and
an external device receiving the first and second physiological signals.

17. The measuring system as claimed in claim 16, wherein the transmittal unit transmits the first and second physiological signals according to a wired transmission technology.

18. The measuring system as claimed in claim 16, wherein the transmittal unit transmits the first and second physiological signals according to a wireless transmission technology.

19. The measuring system as claimed in claim 16, wherein the external device analyzes the first and second physiological signals to generate an analysis result.

20. The measuring system as claimed in claim 16, wherein the external device comprises:
a display unit presenting the analysis result by text or image; and
a storage unit storing the analysis result.
